(19) United States
(12) Patent Application Publication
(10) Pub. No.: US 2006/0008868 A1
(43) Pub. Date: Jan. 12, 2006

(54) Production method of biochemical coal

(75) Inventor: Chien-Chen Chen, Taipei (TW)

Correspondence Address: BRUCE H. TROXELL, SUITE 1404, 5205 LEESBURG PIKE, FALLS CHURCH, VA, 22041, US

(73) Assignee: GREEN TECH Biotechnology Environmental Co., Ltd.

(21) Appl. No.: 10/948,205

(22) Filed: Sep. 24, 2004

(30) Foreign Application Priority Data
Jul. 6, 2004 (TW) 93120214

(51) Int. Cl. C12P 1/00; C12N 5/02

(52) U.S. Cl. 435/41; 435/410

(57) ABSTRACT

A production method of biochemical coal, comprises: a) providing at least a first carbon-containing substance and a second carbon-containing substance; b) pulverizing said first carbon-containing substance and said second carbon-containing substance; c) mixing a first proportion amount of said first carbon-containing substance, a second proportion amount of said second carbon-containing substance and a third proportion amount of a biochemical enzymatic additives to form a mixture; d) having said mixture go through high pressure shaping, conferring it a fixed shape; and e) spraying with a biochemical enzyme group preparation on surface of said mixture conferred fixed shape for air-blocking.

### FIELD OF THE INVENTION

The present invention relates to a production method of biochemical coal fuel, especially relates to a biochemical coal production method by adding biochemical preparation and using high pressure shaping.

### BACKGROUND OF THE INVENTION

In society today, with the progress of industry, the need for fuel coal increases daily whether in transportation, steel making or power generation. But through continuous mining, quality coalmines decrease daily, quality coal is harder to get and expensive. Anthracites on market today mostly are composed from larger content of fixed carbon and smaller content of volatile carbon; though with high heat value, due to low volatile carbon, it has drawbacks of “not ready combustible and burning too hard once ignited” also for the raised environmental consciousness and stricter environmental rules, quite some coals of lower quality turn unusable, such as those with sulfur content over 1%, and lose mining value—relatively making usable coal even less. And coal is ready auto-combustible in stacking, so not stacked compactly, to increase larger space and shipment cost, on top of need of often turning and watering to lower temperature thus rate of spontaneous combustion. And in transport, there is need for special coal piers and ships, and ready dust pollution by wind. So poor quality coals with high sulfur content, high volatile carbon, high fixed carbon, high ash content, low melting point, through specific formulation and biochemical additives converted into environment friendly quality coal, increased volume density without endangering public safety and lower shipment cost are traders urgent problems to solve.

### SUMMARY OF THE INVENTION

The present invention is mainly to provide a production method for biochemical coal. Said method converts poor quality coals such as with high sulfur content, high volatile carbon, high fixed carbon, high ash content, low melting point etc. into quality environment friendly coals with increased volume density, to lower shipment cost and dust pollution of air in transport through specific formulation and biochemical additives.

The present invention secondly is to provide a production method of biochemical coal, said method with special microbe's enzyme group preparation inhibits microbes on the coal surfaces, more over lowers activity of anaerobic microbes, reduces coal into methane marsh gas and blocks air from coals to prevent auto-combustion.

The present invention otherwise is to provide a production method of biochemical coal, said method forms an oxygen-rich membrane on coal surface allowing the coal for low-oxygen combustion, relatively lowering theoretical air amount in combustion and proportion amount of nitrogen oxides in the exhaust after combustion, to lessen air pollution. For the above goals, said production method of biochemical coal comprises: to
a) provide a first carbon-containing substance and a second carbon-containing substance;
b) pulverize said first carbon-containing substance and said second carbon-containing substance;
c) mix a first proportion amount of said first carbon-containing substance, a second proportion amount of said second carbon-containing substance and a third proportion amount of an additive to form a mixture;
d) have said mixture a fixed shape through high pressure shaping, and
e) on surface of said mixture with fixed shape spray a preparation of biochemical enzyme group.

In order for the examiners to further understand and identify the structure, efficacy and method of the present invention, detailed description is given below with diagrams.

### BRIEF DESCRIPTION OF THE DRAWINGS

FIG. 1 is the production flow chart of biochemical coal of the present invention.

FIG. 2 is the block diagram of energy source exchange of the microbe system.

### DESCRIPTION OF THE PREFERRED EMBODIMENT

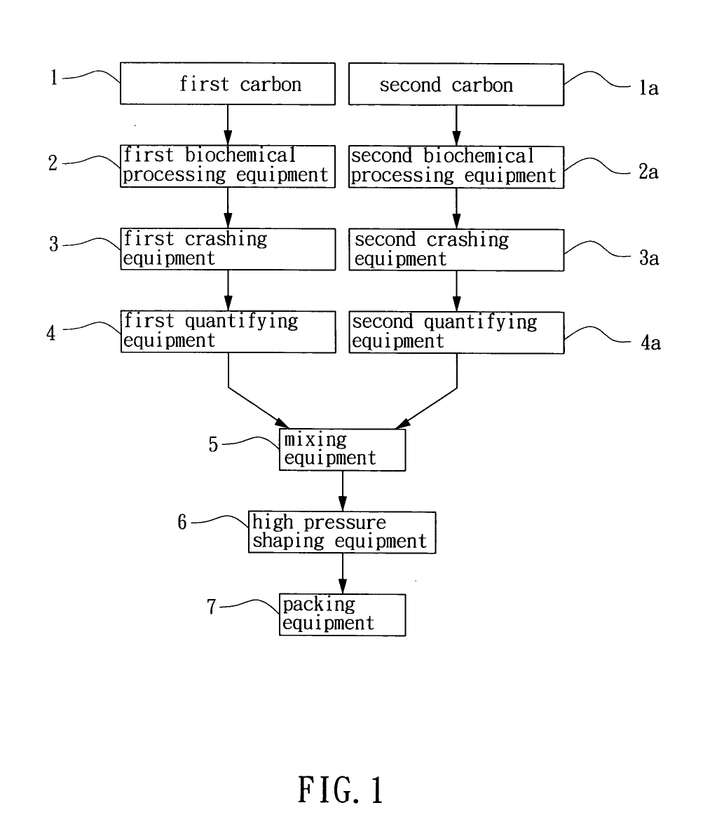

FIG. 1 is the production flow chart of the present invention of biochemical coal. In FIG. 1, step 1 and step 1a are separately to provide a first carbon-containing substance and a second carbon-containing substance, wherein said first carbon-containing substance is anthracite coal or semi-anthracite coal with volatile carbon content below 15%, said second carbon-containing substance is raw coconut shell, palm plant, cane trash, wheat stem, corn stem, cow/sheep pie, Biomass, sow dust, straw, brown coal or others with volatile carbon content (such as over 50%).

And in step 2 and 2a, separately, through a first biochemical processing equipment and a second biochemical processing equipment inhibit unfavorable microbes on surface of said first carbon-containing substance and said second carbon-containing substance, further to reduce reduction by anaerobic microbes of said first carbon-containing substance and said second carbon-containing substance during stacking and shipment.

Further, in step 3 and 3a, using a first crashing equipment and a second crashing equipment separately have said first carbon-containing substance and said second carbon-containing substance go through crashing process, for said first carbon-containing substance and said second carbon-containing substance to be separately pulverized into small powder.

And in step 4 and 4a, according to the features of the final product (such as sulfur content below 1%, heat value over 6000 kcal/kg or volatile carbon proportion amount over 30% etc.), through a first quantifying equipment and a second quantifying equipment for said first carbon-containing substance and said second carbon-containing substance to be analyzed of physical property, and formulated of proportion amount of said first carbon-containing substance and said second carbon-containing substance.

And, step 5 is to provide a mixing equipment, in said mixing equipment, to have the formulated said first carbon-containing substance and said second carbon-containing substance go through mixing, and also in said mixing equipment add in an additive; ref., the action theory of said additive is through high biochemical activity enzyme group with catalytic conversion, for the carbon-containing substance ( such as: fuel coal) to undergo long chain cleavage and better its physical-chemical feature, with quality of coal raised, and for the carbon-containing substance to undergo fast oxidation in combustion and raise the electron transfer speed, and after carbon-containing substance mixing, to form on carbon-containing substance surface oxygen-rich membrane, thus to reduce need for large amount of air with complete combustion, to manage the demand of high efficiency combustion, to efficiently reduce air needed in combustion chamber, and relatively reduce nitrogen oxides discharge, said additive further expedites electron transfer in combustion process, making combustion (oxidation) reaction even more complete, therefore reduce carbon monoxide discharge and raise efficacy of energy source; in preferred embodiment of the present invention, said additive 10 is environment protection enzyme group additive ( BIO-POWER&reg;) for fuel coal, and amount of addition amount to about 0.1% of the mixed said first carbon-containing substance and said second carbon-containing substance.

Subsequently in step 6, have said mixed said first carbon-containing substance, said second carbon-containing substance and said additive sent to a high pressure shaping equipment for high pressure shaping into a fixed shape conferred to the mixture, in preferred embodiment of the present invention, said fixed shape is rectangular cubic (brick-shaped), of course, said fixed shape also could be any known geometrical shape.

Subsequently in step 7, said mixture is sent to a packing equipment, said packing equipment is to have a biochemical air blocking agent sprayed on said mixture surface, for said mixture surface to be air blocked, making said mixture not readily happen auto-combustion and anaerobic microbe's reduction, therefore increase stability in transport and dense stacking. Finally, said mixture sprayed with biochemical enzyme group preparation becomes a biochemical coal brick ref., fuel coal stored stacked for 3 month, especially under tropical room temperature, losing about spontaneous oxidation 10% heat calorie value, losing very great economic efficacy of energy source.

While fuel coal is a natural organic fossil fuel, oxygen not readily pervading fuel coal body in stacking process, in long term closed stacking under high temperature, readily induces activation of anaerobic microbes, having fuel coal reduced into methane marsh gas, causing fuel coal spontaneous combustion (spontaneous ignition) in stack. The present invention uses Oxidative Phosphorylation of Respiratory Chain enzyme group of microbes, its electron transfer is shown in diagram below: 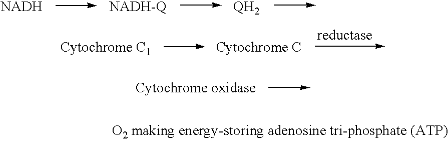
Wherein iron atom of heme group of Cytochrome C is chemically bonded to sulfur atom of methionine and nitrogen atom of histidine, as shown in diagram below:

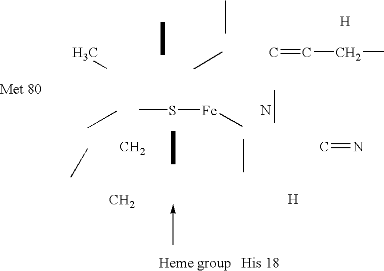

Mitochondria Cytochrome C of tuna cardiac muscle, Cytochrome C₂ of photosynthetic bacteria Rhodosporillum rubrum, and Cytochrome C₅₅₀ of Denitrifying bacteria Paracoccus denitrificans etc. are all conferred with similar chemical bond in this molecular structure.

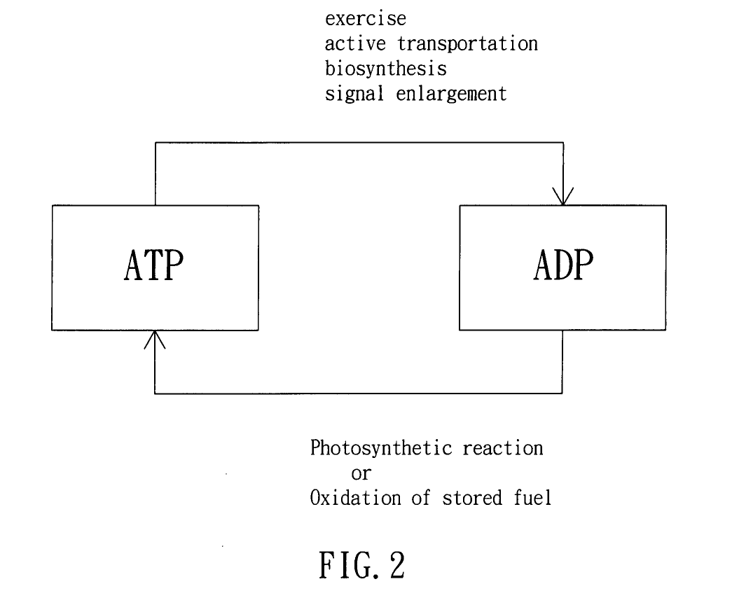

While energy source exchange of microbes system, adopts adenosine tri-phosphate adenosine di-phosphate cycle (ATP-ADP Cycle) (as shown in FIG. 2), under action of chosen microbes enzyme group, the spontaneous oxidation of stored fuel coal turns into ATP storage, therefore efficacy of energy source is not lost; and Oxidative phosphorylation, finally uses oxygen (O₂) as electron acceptor, inhibiting anaerobic bacteria methanization, therefore inhibiting fuel coal auto-combustion.

Therefore in preferred embodiment of the present invention said air blocking agent is a biochemical enzyme, for example: mitochondria Cytochrome C of tuna cardiac muscle, Cytochrome C₂ of photosynthetic bacteria Rhodosporillum rubrum and Cytochrome C₅₅₀ of Denitrifying bacteria Paracoccus denitrificans.

In conclusion, production method of biochemical coal of the present invention is conferred the following advantages: 1) poor quality coals as with high sulfur content, high volatile carbon, high fixed carbon, high ash content, and low melting point etc., through specific formulation and biochemical additive treatment are converted into environment friendly environment protective quality coal, and can be formulated into coals of different specification standards according to the users' demand; 2) using enzyme group preparation to reduce biological oxidation, and making coal blocked from air, excluding coal auto-combustion from happening, further to form an oxygen-rich membrane on coal surface, masking coal combustible in low oxygen, relatively reducing theoretical air amount for combustion, reducing nitrogen oxide proportion amount in exhaust after combustion, reduce air pollution; 3) after high pressure shaping into coal brick, to be shipped with regular container, raising shipping density, without special coal pier and coal ship, therefore lowering transport cost and reduce transport dust pollution due to wind; 4) after high pressure shaping into coal, due to volume density raised, not readily contacting moisture in air, so coal water content not increased, therefore not influencing combustion efficacy of coal; 5) after high pressure shaping into coal brick, making its unit constituent ratio fixed, making coal quality sampling yields a stable value, not like powdery mix coal with big constituent ratio change and hard to sample a standard mean.

Only the abovementioned is just the preferred embodiment of the present invention, and should not limit the range of embodiment of the present invention. Namely all changes and modification according to the claims of the present invention should still belong within the range of patent coverage of the present invention. Hereby the inventor requests sage identification and favorable granting of the examiners as the most hoped for.

While the preferred embodiment of the invention has been set forth for the purpose of disclosure, modifications of the disclosed embodiment of the invention as well as other embodiments thereof may occur to those skilled in the art. Accordingly, the appended claims are intended to cover all embodiments which do not depart from the spirit and scope of the invention.

What is claimed is:

1. A production method of biochemical coal, which comprises:
a) providing a first carbon-containing substance and a second carbon-containing substance;
b) pulverizing said first carbon-containing substance and said second carbon-containing substance;
c) mixing a first proportion amount of said first carbon-containing substance, a second proportion amount of said second carbon-containing substance and a third proportion amount of a additive to form a mixture;
d) having said mixture go through high pressure shaping, conferring it a fixed shape; and
e) on mixture surface of said conferred fixed shape, spraying with a biochemical enzyme group preparation for air blocking.

2. The production method of biochemical coal as stated in claim 1, wherein said first carbon-containing substance and second carbon-containing substance is formed through a biochemical treatment, and said biochemical treatment is to inhibit surface microbes on said first carbon-containing substance and second carbon-containing substance.

3. The production method of biochemical coal as stated in claim 1, wherein said first carbon-containing substance is anthracite coal with volatile carbon content lower than 15%.

4. The production method of biochemical coal as stated in claim 1, wherein said first carbon-containing substance is a coal with sulfur content over 1%.

5. The production method of biochemical coal as stated in claim 1, wherein said second carbon-containing substance is coconut shell.

6. The production method of biochemical coal as stated in claim 1, wherein said third proportion amount is amounted to about 0.1% of said mixture.

7. The production method of biochemical coal as stated in claim 1, wherein said additive is fuel coal's environment protection enzyme group additive (BIO-POWER&reg;).

8. The production method of biochemical coal as stated in claim 1, wherein said fixed shape is a rectangular cubic.

9. The production method of biochemical coal as stated in claim 1, wherein said biochemical enzyme group preparation is a biochemical enzyme.

10. The production method of biochemical coal as stated in claim 1, wherein the kinds of said biochemical enzyme group preparation are microbe enzymes: Xanthomonas campestris (Xanthan gum), Guar gum, Gum Arabic, Gum Tragacanth, Alginate (P. aeruginosa), Carragenan (extract of red algae), Pectin, Chitosan, Cellulose, Starch, milk, Soy protein, Alginate, viscose: CMC, PVP.
